(19) United States
(12) Patent Application Publication
(10) Pub. No.: US 2008/0208019 A1
(43) Pub. Date: Aug. 28, 2008

(54) Modified Pulse Oximetry Technique For Measurement Of Oxygen Saturation In Arterial And Venous Blood

(75) Inventor: Meir Nitzan, Beit El (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: JERUSALEM COLLEGE OF TECHNOLOGY, Jerusalem (IL)

(21) Appl. No.: 11/677,585

(22) Filed: Feb. 22, 2007

(51) Int. Cl. A61B 5/00

(52) U.S. Cl. 600/323

(57) ABSTRACT

A method for the measurement of oxygen saturation in arterial blood SaO₂ by means of pulse oximetry without calibration is described. Photoplethysmography (PPG) is measured in three wavelengths in the infrared and for each PPG curve the relative change in light transmission is obtained. Two equations, each relating SaO₂ to the ratio of the relative changes in light transmission for two wavelengths, using the values of the extinction coefficients of oxygenated and deoxygenated hemoglobin for the three wavelengths, enable the determination of SaO₂, assuming linear dependence of the optical path-length on the wavelength, for the three wavelengths in the infrared. Similar technique for the determination of oxygen saturation in venous blood from changes in light transmission due to changes in venous blood volume is suggested

### FIELD AND BACKGROUND OF THE INVENTION

### 1.1. Arterial and Venous Oxygen Saturation

Transfer of oxygen from the lungs to the tissue cells is done mainly via the hemoglobin molecules in the red blood cells. Total oxygen content in blood includes the oxygen connected to hemoglobin and the dissolved oxygen in arterial plasma, measured by the partial pressure PaO₂ of the dissolved oxygen. Oxygen saturation SO₂ is the ratio of oxygenated hemoglobin to total hemoglobin (SO₂=HbO₂/(HbO₂+Hb)), and its value in the arterial blood, SaO₂, depends on the adequacy of the ventilation and respiratory function. SaO₂ is related to the partial oxygen pressure PaO₂ by S-shaped curve: SaO₂ increases steeply with PaO₂ for PaO₂ values between 10 and 50 mmHg (at PaO₂ of 50 mmHg SaO₂ is about 80%), then increases moderately. Normal values of SaO₂ are 94-99%. Assessment of SaO₂ is mainly important for clinical evaluation of proper respiratory function.

Most of the hemoglobin in venous blood is still oxygenated: normal values of the oxygen saturation in the peripheral venous blood, SvO₂ are 70-80%. SvO₂ also has physiological and clinical significance, as lower blood flow to the tissue results in higher utilization of the oxygen in the blood and lower value of SvO₂. The assessment of blood supply to the skin in limbs or other organ can provide information on the adequacy of skin blood flow. Low values of skin blood flow can also indicate the occurrence of shock or cardiac failure, in which blood flow is diverted from the peripheral circulation towards more vital organs. Note that, in contrast to the routine use of pulse oximetry for SaO₂ measurement, no accepted method for the measurement of SvO₂ is available.

### 1.2 Optical Measurement of Arterial Oxygen Saturation

The different light absorption spectrum for oxygenated and de-oxygenated hemoglobin enables the noninvasive assessment of oxygen saturation in the blood. The transmission of light through a given tissue depends on the light absorption and the light scattering coefficients of the various components of the tissue, including the arterial and venous blood. Transmission of light depends on the light scattering because the latter increases the path-length of the light (by a factor of 5-10). In order to isolate the contribution of the arterial blood to the light absorption, the absorption of light of several wavelengths through the ear-lobe was measured after heating the tissue to 41° C., which results in “arterialization” of the blood (Mendelson, 1988). At present the usual technique for the isolation of the contribution of the arterial blood to the light absorption is based on photoplethysmography (PPG)—the measurement of light absorption changes due to the cardiac induced blood volume changes (Yoshiya, 1980). Since the PPG signal originates from the arterial blood volume increase during systole, the measurement of the PPG signal in several wavelengths—pulse oximetry—enables the assessment of the oxygen saturation in the arterial blood, SaO₂.

### 1.3. The PPG Signal and its Origin

Photoplethysmography (PPG) is the recording of tissue blood volume changes by the measurement of light absorption in the tissue. The PPG probe consists of a light source emitting light into the tissue and a photodetector measuring the light transmitted through the tissue. During systole blood is ejected from the left ventricle into the peripheral vascular system, thereby increasing the arterial blood content, and consequently decreasing the light intensity transmitted through the tissue. The PPG signal is shown in FIG. 2. The decrease in the transmitted light intensity during systole originates from the cardiac induced increase in the arterial blood volume during systole. The maximal value of the PPG signal (BL in FIG. 2) is proportional to the light irradiance transmitted through the tissue at end-diastole, when the tissue blood volume is minimal. The amplitude AM of the PPG signal is related to the maximal arterial blood volume change during systole ΔVₐ. It can be shown (Babchenko et al. 2001) that since in general AM<<BL,

AM/BL=αₐΔVₐ (1)

where αₐ is the effective absorption coefficient of the arterial blood, which is a function of the absorption and scattering attenuation constants. In general the PPG signal is presented in inverted form (FIG. 3) so that increase in the PPG signal corresponds to increase in tissue blood volume.

### 1.4. Theory of Pulse Oximetry

The theory of deriving arterial oxygen saturation from the PPG signals at two wavelengths is described in several articles (see Wieben, 1997, Mannheimer et al, 1997, Nitzan et al, 2000). The transmitted light intensity, Iₜ, through a sample of hemolized blood is given by the Beer-Lambert Law:

Iₜ=I₀ exp(−αd) (2)

where I_{α} is the incident light intensity, α is the absorption constant of the blood, and d is the width of the tissue sample. The transmitted light intensity, Iₜ, through a tissue sample which includes vessels with whole blood is given by:

Iₜ=I₀ exp(−αl−εcl) (3)

where l is the effective optical path-length, which is higher than d because of scattering in the tissue, α is the absorption constant of the tissue, c is the concentration of the blood in the tissue, and ε is the extinction coefficient of the blood, defined as the absorption constant of the blood divided by the concentration of the blood in the tissue.

During systole the tissue blood volume increases and consequently the light transmission through the tissue decreases. Photoplethysmography (PPG) is the measurement of the oscillatory changes in light transmission through tissue due to the cardiac induced blood volume changes in the tissue. If I_{S} is the light transmission through the tissue during the maximal increase in tissue blood volume and I_{D} is the light transmitted through the tissue during end diastole (when the tissue blood volume has its minimal value), then

I_{S}=I_{D} exp(−εₐΔcₐl)

ln(I_{D}/I_{S})=εₐΔcₐl (4)

where εₐ is the extinction coefficient for the arterial blood, and Δcₐ is the increase of blood concentration (in the tissue) due to the maximal systolic increase of blood volume. For small blood volume changes ΔIₐ=I_{D}−I_{S}<<I_{S}, and ln(I_{D}/I_{S}) can be approximated by ΔIₐ/I_{S}. If the light transmission is measured for two wavelengths λ₁ and λ₂, then

(ΔIₐ/I_{S})₁=εₐ₁Δcₐ₁l₁; (ΔIₐ/I_{S})₂εₐ₂Δcₐ₂l₂ (5)

and the ratio R, defined by

$$R = \frac{{( Δ {I}_{a} / {I}_{S} )}_{1}}{{( Δ {I}_{a} / {I}_{S} )}_{2}} \qquad ( 6 )$$

satisfies the equation

$$R \approx \frac{{ɛ}_{a 1} {l}_{1}}{{ɛ}_{a 2} {l}_{2}} \qquad ( 7 a )$$

assuming that the difference in the blood concentration change Δcₐ between the two wavelengths can be neglected (Δcₐ₁≈Δcₐ₂). Note that the last assumption is not always satisfied if the penetration depth for the two wavelengths is different, which can especially occur in reflection PPG. If the two wavelengths are close to each other then the difference of the effective optical path-length is small and

$$R \approx \frac{{ɛ}_{a 1}}{{ɛ}_{a 2}} \qquad ( 7 b )$$

The relationship between the ratio R—the measured parameter—and the oxygen saturation SaO₂ can be derived from the decomposition of the extinction coefficient ε into its two components, the extinction coefficients for oxygenated blood εₒ and for deoxygenated blood ε_{d}:

$$\begin{matrix}ɛ = {ɛ}_{o} Sa {O}_{2} + {ɛ}_{d} ( 1 - Sa {O}_{2} ) = {ɛ}_{d} + Sa {O}_{2} ( {ɛ}_{o} - {ɛ}_{d} ) \\ Then & ( 8 ) \\ R = \frac{{ɛ}_{d 1} + Sa {O}_{2} ( {ɛ}_{o 1} - {ɛ}_{d 1} )}{{ɛ}_{d 2} + Sa {O}_{2} ( {ɛ}_{o 2} - {ɛ}_{d 2} )} \\ and & ( 9 ) \\ Sa {O}_{2} = \frac{{ɛ}_{d 1} - R {ɛ}_{d 2}}{R ( {ɛ}_{02} - {ɛ}_{d 2} ) + ( {ɛ}_{d 1} - {ɛ}_{01} )} & ( 10 )\end{matrix}$$

In order to have a higher difference in light transmittance between the two wavelengths, commercial pulse oximeters choose one of the wavelengths in the infrared region, above the isosbestic wavelength (805 nm) and the other in the red region, where the difference in the extinction coefficient between oxygenated and deoxygenated blood is maximal. However, for this choice, the red light scattering constant significantly differs from that of the infrared light resulting in non-negligible difference in optical path-lengths for the two wavelengths. From Equation (7a)

$$R ( {l}_{1} / {l}_{2} ) = \frac{{ɛ}_{o 1}}{{ɛ}_{o 2}} \qquad ( 11 )$$

By replacing R in Eqs. 9-10 with R(l₂/l₁), Equation (10) becomes

$$Sa {O}_{2} = \frac{{ɛ}_{d 1} - R ( {l}_{1} / {l}_{2} ) {ɛ}_{d 2}}{R ( {l}_{1} / {l}_{2} ) ( {ɛ}_{02} - {ɛ}_{d 2} ) + ( {ɛ}_{d 1} - {ɛ}_{01} )} \qquad ( 12 )$$

Taking λ₁ as the lower wavelength, l₁ is higher than l₂ due to the higher scattering for the lower wavelength. By using the measured higher value of R instead of the lower value of R(l₂/l₁), the calculated value of SaO₂ is lower than its real value.

Due to the difference between the optical properties of the red and infrared light, the clinical parameter oxygen saturation SaO₂ cannot be derived from the measured parameter R and Equation (10). The actual relationship between the measured parameter R and oxygen saturation SaO₂ is achieved for each pulse oximeter sensor by calibration (Schowalter, 1997): R is measured in several persons simultaneously with in vitro SaO₂ measurement in extracted arterial blood by means of co-oximeter which is the gold-standard for SaO₂ measurements. For each person R and SaO₂ measurements are taken for several values of SaO₂, achieved by changing the partial pressure of oxygen in the inspired air. The table of the simultaneous measurements of R and SaO₂ provides the required calibration for the derivation of the clinical parameter oxygen saturation SaO₂ from the measured parameter R. The reliability of the calibration is based on the assumption that l₂/l₁ does not change between different persons and different physiological and clinical situations. The validity of this assumption is limited and deviations from this assumption are the likely origin of the inherent inaccuracy of the pulse oximetry technique for the assessment of the oxygen saturation, SaO₂, in the arterial blood.

In the description of the pulse oximetry method presented above, R was defined as the ratio of the ratios ΔIₐ/I_{S} for the two wavelengths R can also be defined as the ratio of two values of a parameter related to change in light transmission for the two wavelengths, and this parameter can be different than ΔIₐ/I_{S}. It can be chosen as (I_{D}/I_{S}) (as in U.S. Pat. Nos. 4,773,422 and 4,167,331) or the derivative of I divided by I (as in U.S. Pat. No. 6,505,060). As stated above, due to the higher scattering for the lower wavelength, l₁>l₂ for λ₁<λ₂, and by using the measured higher value of R instead of the lower value of R(l₂/l₁), the calculated value of SaO₂ is lower than its real value. If the two wavelengths are close to each other, l₂/l₁ is expected to be about 1 and SpO₂ can be derived from Equation (10) without calibration. This was shown in our study (Nitzan et al 2000), in which we measured SpO₂, using pulse oximetry based on two infrared wavelengths (767 and 811 nm) and Equation (10), without calibration. The SpO₂ values were lower than that of the available pulse oximeters using red and infrared light, probably because the limitation of the assumption of l₂/l₁=1. The accuracy of the technique increased by using Equation (12) and assessing (l₂/l₁) value from published data (like that of Duncan et al. 2000 for the forearm, (l₂/l₁)=0.97) but since l₂/l₁ is not known for the specific examination the accuracy is still limited.

Oxygen saturation in the peripheral venous blood SvO₂ also has physiological and clinical significance, as described above, but in contrast to the routine use of pulse oximetry to SaO₂ measurement, no accepted method for the measurement of SvO₂ is available. Similar to pulse oximetry which utilizes the PPG changes (corresponding to arterial blood volume changes) in the heart rate for the evaluation of the arterial oxygen saturation, we measured (Nitzan et al. 2000) venous oxygen saturation in the blood accumulated in the veins after applying to the arm pressure higher than venous blood pressure. The use of two wavelengths of similar value of l₂/l₁ is essential because blood extracted from big veins does not necessarily has the same value of oxygen saturation as that of small veins in specific skin site and therefore calibration cannot be used. SvO₂ can also be measured by using the spontaneous blood volume changes in the respiratory rate and in lower frequencies (of about 0.1 Hz and about 0.02 Hz) (Nitzan et al. 1994). We found that the blood volume changes in very-low-frequency are mainly attributed to tissue venous blood volume changes (Nitzan et al 1996).

### 1.5. The Accuracy of Arterial Oxygen Saturation Measurement by Pulse Oximetry

The accuracy of SaO₂ measurement by pulse oximetry (SpO₂) is obtained by comparing the SpO₂ measurement to SaO₂ measurement in extracted arterial blood by means of co-oximeter, which is the gold-standard for SaO₂ measurements. The accuracy of SpO₂ as declared by the manufacturers is 2%, say the standard deviation of the SpO₂ and co-oximeter measurements is 2%, which means that for 5% of the examinations the deviation from the gold-standard is 4%. For SaO₂ values below 80% the accuracy is even lower. Such accuracy is acceptable for the present main use of pulse oximetry; say monitoring arterial oxygen saturation during surgical operations and at intensive care units, in order to check qualitatively that no adverse change happened to the patient. In these cases detection of 3-4% decrease in SaO₂ is required, and in general this task can be properly performed by the available pulse oximeter monitors. However, in some medical units, where respiratory function assessment is required, higher accuracy of SaO₂ measurement is needed. In some studies where accurate oxygenation measurement was required invasive PaO₂ measurement was used instead of noninvasive SpO₂ measurement. As an example, Seccombe et al (2004) examined the effect of lower-than-normal oxygen pressure in breathed air on lung patients, by measuring invasively PaO₂ in their arterial blood, for assessing their potential response to flight environment.

Note that in general the range of SaO₂ values is 80-100%, so that standard deviation of 2% is not small relative to the range values. In particular the range of normal values of SaO₂ is 94-99%, so that pulse oximetry in its present configuration is not useful for assessing SaO₂ in physiological studies of the normal range and for sports medicine.

The low accuracy of SpO₂ measurement probably originates from the requirement for calibration. As stated above, the derivation of the oxygen saturation SaO₂ from the measured ratio R cannot be achieved directly from Equation (10) and together with the values of the extinction coefficients for oxygenated blood εₒ and for deoxygenated blood ε_{d} because l₁ and l₂ are not equal. Equation (12) cannot be used because l₁/l₂ is not known. The calibration process assumes constant value of l₁/l₂ for all persons undergoing examination, independent on the clinical variables, which is not always achieved.

Furthermore, the assumption that the difference in the blood concentration change Δcₐ, between the two wavelengths can be neglected (Δcₐ₁≈Δcₐ₂) is not achieved if the penetration depth for the two wavelengths is different and the tissue is not homogenous. In our studies we found (Nitzan et al. 2000) that the significance of these two phenomena can be reduced, if the two wavelengths will be chosen close to one another so that they will be of similar path-length and similar penetration depth. By using Equation (12) and assessing (l₂/l₁) value from published data (see above), the technique provides SpO₂ value with no need for calibration. The technique requires the use of the values of the extinction coefficients of oxygenated and deoxygenated hemoglobin for the three wavelengths. The accuracy of the technique is, however still limited since l₂/l₁ is not known for the specific vascular setup and blood distribution in tissue for each examination. Accordingly, there is a need for innovative improvement of pulse oximetry in order to obtain accurate non-invasive technique for the monitoring of the oxygen saturation in arterial blood, SaO₂.

Furthermore, there is a need to modify the pulse oximetry technique so that it will be appropriate to measure the oxygen saturation in venous blood, SvO₂.

### SUMMARY OF THE INVENTION

The present invention is a pulse oximetry technique for measurement of oxygen saturation in arterial or venous blood.

According to the teachings of the present invention there is provided, a method for the measurement of oxygen saturation in arterial blood, the method comprising: (a) applying to the skin an apparatus including: (i) light-sources for generating light of three peak wavelengths λ₁, λ₂ and λ₃; (ii) a detector arrangement, and (iii) an electronics unit, the apparatus configured to detect, for each light-source, the transmitted light through the tissue; (b) for each light source, deriving a PPG curve; (c) deriving from the three PPG curves a parameter related to the relative change in light transmission for each of the three wavelengths, and the ratio R₁₂ and R₁₃ of that parameter for the two wavelengths λ₁ and λ₂ and for the two wavelengths λ₁ and λ₃, respectively; and (d) determining the oxygen saturation in arterial blood, SaO₂, from the solution of three equations including:
a. relationship between SaO₂ and R₁₂, which includes the optical path-lengths l₁ and l₂,
b. relationship between SaO₂ and R₁₃, which includes the optical path-lengths l₁ and l₃;
c. relationship between small changes in the path-length l and small changes in the wavelength λ,
   where the first two equations include the values of the extinction coefficient for the three peak wavelength for oxygenated blood εₒ, and for decoxygenated blood ε_{d}.

There is also provided according to the teachings of the present invention, a method for the measurement of oxygen saturation in arterial blood, the method comprising: (a) applying to the skin an apparatus including: (i) light-sources for generating light of three peak wavelengths λ₁, λ₂ and λ₃; (ii) a detector arrangement; and (iii) an electronics unit, the apparatus configured to detect, for each light-source, the transmitted light through the tissue; (b) for each light-source, determining the mean extinction coefficients over the spectrum band of the light-source emitted light for oxygenated blood εₒ and for deoxygenated blood ε_{d}; (c) for each light-source, deriving a PPG curve; (d) deriving from the three PPG curves a parameter related to the relative change in light transmission for each of the three wavelengths, and the ratio R₁₂ and R₁₃ of that parameter for the two wavelengths λ₁ and λ₂ and for the two wavelengths λ₁ and λ₃, respectively; and (e) determining the oxygen saturation in arterial blood, SaO₂, from the solution of three equations including:
a. relationship between SaO₂ and R₁₂, which includes the optical path-lengths l₁ and l₂;
b. relationship between SaO₂ and R₁₃, which includes the optical path-lengths l₁ and l₃;
c. relationship between small changes in the path-length l and small changes in the wavelength λ.
   where the first two equations include the values of the mean extinction coefficient for the three light sources for oxygenated blood εₒ and for deoxygenated blood ε_{d}.

According to a further feature of the present invention, the relationship between SaO₂ and R₁₂, which includes the optical path-lengths l₁ and l₂ is

$$Sa {O}_{2} = \frac{{ɛ}_{d 1} - {R}_{12} ( {l}_{1} / {l}_{2} ) {ɛ}_{d 2}}{{R}_{12} ( {l}_{1} / {l}_{2} ) ( {ɛ}_{02} - {ɛ}_{d 2} ) + ( {ɛ}_{d 1} - {ɛ}_{01} )}$$

According to a further feature of the present invention, the relationship between SaO₂ and R₁₃, which includes the optical path-lengths l₁ and l₃ is

$$Sa {O}_{2} = \frac{{ɛ}_{d 1} - {R}_{13} ( {l}_{1} / {l}_{3} ) {ɛ}_{d 3}}{{R}_{13} ( {l}_{1} / {l}_{3} ) ( {ɛ}_{02} - {ɛ}_{d 3} ) + ( {ɛ}_{d 1} - {ɛ}_{01} )}$$

According to a further feature of the present invention, the three peak wavelengths are in the infrared region.

According to a further feature of the present invention, the relationship between small changes in the path-length l and small changes in the wavelength λ is a linear relationship.

According to a further feature of the present invention, the parameter related to the relative change in light transmission is selected from the group comprising:
a. (I_{D}−I_{S})/I_{S}, where I_{D} is the maximal light transmission and I_{S} is the minimal light transmission;
b. [I(t₁)−I(t₂)]/I(t₂), where I(t₁) and I(t₂) are light transmission values at two time points, t₁ and t₂, along the rise-time of the PPG pulse;
c. ln (I_{D}/I_{S});
d. ln [I(t₁)/I(t₂)], where t₁ and t₂ are two time points along the rise-time of the PPG pulse;
e. (dI(t)/dt)/I(t) at some point along the rise-time of the PPG pulse; and
f. mean of the values of (dI(t)/dt)/I(t) at some points along the rise-time of the PPG pulse.

According to a further feature of the present invention, the light sources are selected from the group comprising: light emitting diodes, light emitting diodes with filter and laser diodes.

There is also provided according to the teachings of the present invention, a method for the measurement of oxygen saturation in tissue venous blood, the method comprising: (a) applying to the skin an apparatus including: (i) light-sources for generating light of three peak wavelengths λ₁, λ₂ and λ₃; (ii) a detector arrangement; and (iii) an electronics unit, the apparatus configured to detect, for each light-source, the transmitted light through the tissue; (b) for each light source, deriving a curve of light transmission through the tissue while venous blood volume changes; (c) deriving from the three light transmission curves a parameter related to the relative change in light transmission for each of the three wavelengths, and the ratio R₁₂ and R₁₃ of that parameter for the two wavelengths λ₁ and λ₂ and for the two wavelengths λ₁ and λ₃, respectively; and (d) determining the oxygen saturation in venous blood, SvO₂, from the solution of three equations including:
a. relationship between SaO₂ and R₁₂, which includes the optical path-lengths l₁ and l₂;
b. relationship between SaO₂ and R₁₃, which includes the optical path-lengths l₁ and l₃;
c. relationship between small changes in the path-length l and small changes in the wavelength λ,
   where the first two equations include the values of the extinction coefficient for the three peak wavelength for oxygenated blood εₒ and for deoxygenated blood ε_{d}.

There is also provided according to the teachings of the present invention, a method for the measurement of oxygen saturation in tissue venous blood, the method comprising. (a) applying to the skin an apparatus including: (i) light-sources for generating light of three peak wavelengths λ₁, λ₂ and λ₃; (ii) a detector arrangement; and (iii) an electronics unit, the apparatus configured to detect, for each light-source, the transmitted light through the tissue; (b) for each light-source, determining the mean extinction coefficients over the spectrum band of the light-source emitted light for oxygenated blood εₒ and for deoxygenated blood ε_{d}; (c) for each light-source, deriving a curve of light transmission through the tissue while venous blood volume changes; (d) deriving from the three light transmission curves a parameter related to the relative change in light transmission for each of the three wavelengths, and the ratio R₁₂ and R₁₃ of that parameter for the two wavelengths λ₁ and λ₂ and for the two wavelengths λ₁ and λ₃, respectively; and (e) determining the oxygen saturation in venous blood, SvO₂, from the solution of three equations including:
a. relationship between SaO₂ and R₁₂, which includes the optical path-lengths l₁ and l₂;
b. relationship between SaO₂ and R₁₃, which includes the optical path-lengths l₁ and l₃;

c. relationship between small changes in the path-length l and small changes in the wavelength λ.

where the first two equations include the values of the mean extinction coefficient for the three light sources for oxygenated blood εₒ and for deoxygenated blood ε_{d}.

According to a further feature of the present invention, the relationship between SvO₂ and R₁₂, which includes the optical path-lengths l₁ and l₂ is

$$Sv {O}_{2} = \frac{{ɛ}_{d 1} - {R}_{12} ( {l}_{1} / {l}_{2} ) {ɛ}_{d 2}}{{R}_{12} ( {l}_{1} / {l}_{2} ) ( {ɛ}_{02} - {ɛ}_{d 2} ) + ( {ɛ}_{d 1} - {ɛ}_{01} )}$$

According to a further feature of the present invention, the relationship between SvO₂ and R₁₃, which includes the optical path-lengths l₁ and l₃ is

$$Sv {O}_{2} = \frac{{ɛ}_{d 1} - {R}_{13} ( {l}_{1} / {l}_{3} ) {ɛ}_{d 3}}{{R}_{13} ( {l}_{1} / {l}_{3} ) ( {ɛ}_{02} - {ɛ}_{d 3} ) + ( {ɛ}_{d 1} - {ɛ}_{01} )}$$

According to a further feature of the present invention, the three peak wavelengths are in the infrared region.

According to a further feature of the present invention, the relationship between small changes in the path-length l and small changes in the wavelength λ is linear relationship.

According to a further feature of the present invention, the parameter related to the relative change in light transmission is selected from the group comprising:
a. (I_{D}−I_{S})/I_{S}, where I_{D} is the maximal light transmission and I_{S} is the minimal light transmission;
b. [I(t₁)−I(t₂)/I(t₂), where I(t₁) and I(t₂) are light transmission values at two time points, t₁ and t₂, along the rise-time of the PPG pulse;
c. ln (I_{D}/I_{S});
d. ln [I(t₁)/I(t₂)], where t₁ and t₂ are two time points along the rise-time of the PPG pulse;
e. (dI(t)/dt)/I(t) at some point along the rise-time of the PPG pulse; and
f. mean of the values of (dI(t)/dt)/I(t) at some points along the rise-time of the PPG pulse.

According to a further feature of the present invention, the light sources are selected from the group comprising: light emitting diodes, light emitting diodes with filter and laser diodes.

According to a further feature of the present invention, the change in venous blood volume is achieved by closing the veins conveying blood from the tissue, by use of a pressure cuff.

According to a further feature of the present invention, the change in venous blood volume is spontaneous.

According to a further feature of the present invention, the spontaneous change in venous blood volume is at the respiratory rate.

According to a further feature of the present invention, the spontaneous change in venous blood volume is at a frequency of about 0.1 Hz.

According to a further feature of the present invention, the spontaneous change in venous blood volume is at a frequency of 0.01-0.04 Hz

### BRIEF DESCRIPTION OF THE DRAWINGS

The invention is herein described, by way of example only, with reference to the accompanying drawings, wherein:

FIG. 1 is a graph showing the extinction coefficients for oxygenated blood (HbO₂) and for deoxygenated blood (Hb) as a function of the wavelength.

FIG. 2 is a plot of a PPG signal against time showing variations at the heart rate, wherein maximal intensity of transmitted light occurs at end-diastole when the tissue blood volume is minimum and the light transmitted through the tissue is at maximal intensity, and wherein. BL is the baseline, AM is the amplitude; and P is the period.

FIG. 3 is a plot showing a series of PPG pulses inverted.

### DESCRIPTION OF THE PREFERRED EMBODIMENTS

In the current invention, the transmission of light through the finger of three adjacent wavelengths in the infrared region, λ₁, λ₂, and λ₃, will be measured for the assessment of SaO₂. Equation (12) will be used for the two pairs λ₁, λ₂, and λ₃:

$$Sa {O}_{2} = \frac{{ɛ}_{d 1} - {R}_{12} ( {l}_{1} / {l}_{2} ) {ɛ}_{d 2}}{{R}_{12} ( {l}_{1} / {l}_{2} ) ( {ɛ}_{02} - {ɛ}_{d 2} ) + ( {ɛ}_{d 1} - {ɛ}_{01} )} \\ Sa {O}_{2} = \frac{{ɛ}_{d 1} - {R}_{13} ( {l}_{1} / {l}_{3} ) {ɛ}_{d 3}}{{R}_{13} ( {l}_{1} / {l}_{3} ) ( {ɛ}_{02} - {ɛ}_{d 3} ) + ( {ɛ}_{d 1} - {ɛ}_{01} )} \qquad ( 13 )$$

where R₁₂ and R₁₃ are the ratio of the relative changes in light transmission ΔIₐ/I_{S} for the two wavelengths λ₁ and λ₂ and the two wavelengths λ₁ and λ₃, respectively, and l₁, l₂ and l₃ are the path-lengths of the three wavelengths, λ₁, λ₂, and λ₃, respectively.

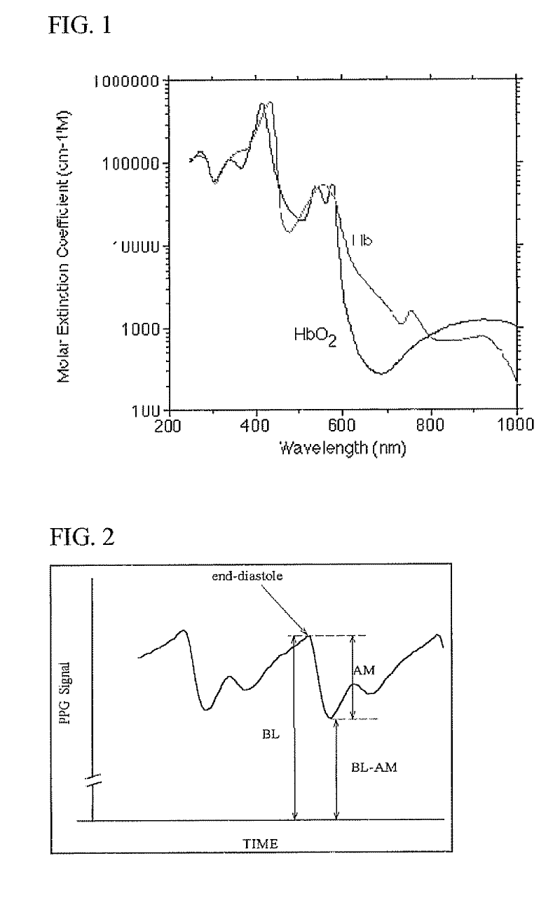

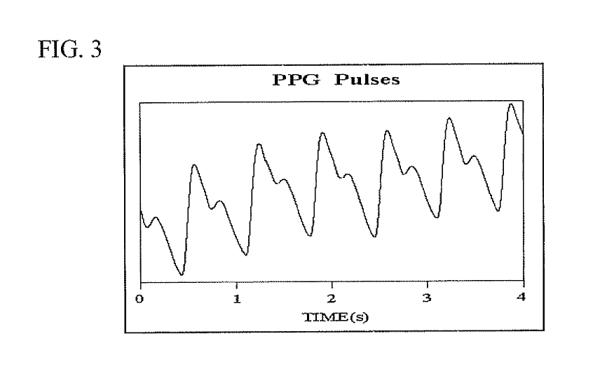

R₁₂ and R₁₃ of Equation (13) can be measured and the extinction coefficients for the three wavelengths can be found for oxygenated blood εₒ and for deoxygenated blood ε_{d} in the literature. FIG. 1 shows a graph of the extinction coefficients for oxygenated blood (HbO₂) and for deoxygenated blood (Hb) as a function of the wavelength. However, in order to determine SaO₂, it is necessary to find l₁/l₂ and l₁/l₃. Since the dependence of scattering on the wavelength is smooth, the relationship between the path-length l and the wavelength λ is also smooth, and one can assume linear relationship between small changes in the path-length l and small changes in the wavelength λ.

l=l₀+Const(λ−λ₀) (14)

which yields the following relationship between l₁/l₂ and l₁/l₃:

(λ₂−λ₁)/(λ₃−λ₁)=(l₂−l₁)/(l₃−l₁)=(l₂/l₁−1)/(l₃/l₁−1) (15)

From the three equations, Equations (13) and Equation (15), SaO₂ can be determined without prior information on l₁/l₂ and l₁/l₃, since we have three equations in three unknowns.

The measurement of pulse oximetry by three wavelengths or more has been already suggested for accurate measurement of oxygen saturation in the presence of carboxyhemoglobin and methemoglobin (U.S. Pat. Nos. 5,413,100, 4,167,331). The extinction coefficient of carboxyhemoglobin and of methemoglobin differs from that of oxygenated hemoglobin and deoxygenated hemoglobin: carboxyhemoglobin is almost transparent in the infrared region and methemoglobin has higher absorption for the red light and lower absorption for the infrared region. Our technique is different since it is based on Equations (13) and (15).

U.S. Pat. No. 6,801,799 by Mendelson suggests pulse oximeter of three wavelengths, one in red and two in infrared. The latter two wavelengths are used for compensating for changes in contact pressure or site-to-site variations. The technique suggested by Mendelson includes several detectors arranged around the light sources in closed path, while our technique requires only one detector and is based on Equations (13) and (15). Mannheimer in U.S. Pat. No. 5,782,756 utilizes three wavelengths for compensating for variations caused by scattering differences at different wavelengths. This is done by deriving SaO₂ by means of two pairs of wavelengths in the regular technique (say Equation (10) or similar equation, including calibration for the determination of the relationship between SaO₂ and R). The first pair of wavelengths provides the SaO₂ initial assessment and the other SaO₂ value, derived from the other pair of wavelengths, is used for correcting the initial assessment. In our technique the two pairs of PPG measurement are utilized, together with a third equation, for the determination of SaO₂.

The technique suggested by Mannheimer in U.S. Pat. No. 5,782,756 differs from our technique in three elements.
The algorithms are different
Calibration is required for the technique suggested by Mannheimer, while our technique needs no calibration. On the other hand, in our technique the mean extinction coefficients over the spectrum band of the light-source emitted light has to be derived for the three wavelengths both for oxygenated blood εₒ and for deoxygenated blood ε_{d}, while in Mannheimer's technique it is not required
The choice of the suitable three wavelengths is different. Mannheimer suggests preferred wavelength combinations based on overlap in penetration of light and sufficient difference in extinction coefficient between the wavelengths. In our technique we suggest using light of wavelengths in the infrared region, as well as small difference between the wavelengths, so that the dependence of the path-length l and the wavelength λ is linear.

The suggested technique is also suitable for SvO₂ assessment, by measuring light transmission change in infrared light of three wavelengths, originated from change in venous blood volume. As noted above, the change in venous blood volume can be a result of vein occlusion after applying to the arm pressure higher than venous blood pressure but lower than arterial blood pressure, or can be spontaneous, in respiration rate due to the respiratory effect on blood vessels or in lower frequency, due to autonomic nervous system activity.

The preferred pulse oximeter is a device, applied to the skin, which includes single LED or several LEDs of three peak wavelengths in the infrared region and a detector which can detect, for each wavelength, the transmitted light through the tissue. For each LED, the mean extinction coefficients over the spectrum band of the LED emitted light is derived for oxygenated blood εₒ and for deoxygenated blood ε_{d}. For each wavelength a PPG curve is obtained. From the three PPG curves a parameter related to the relative change in light transmission is obtained for each of the three wavelengths, and the ratio R₁₂ and R₁₃ of that parameter for the two wavelengths λ₁ and λ₂ and for the two wavelengths λ₁ and λ₃, respectively, is derived. SaO₂ is determined from the pair of equations.

$$Sa {O}_{2} = \frac{{ɛ}_{d 1} - {R}_{12} ( {l}_{1} / {l}_{2} ) {ɛ}_{d 2}}{{R}_{12} ( {l}_{1} / {l}_{2} ) ( {ɛ}_{02} - {ɛ}_{d 2} ) + ( {ɛ}_{d 1} - {ɛ}_{01} )}$$

$$and$$

$$Sa {O}_{2} = \frac{{ɛ}_{d 1} - {R}_{13} ( {l}_{1} / {l}_{3} ) {ɛ}_{d 3}}{{R}_{13} ( {l}_{1} / {l}_{3} ) ( {ɛ}_{02} - {ɛ}_{d 3} ) + ( {ɛ}_{d 1} - {ɛ}_{01} )}$$

using linear relationship between small changes in the path-length l and small changes in the wavelength λ.

In another preferred embodiment laser diodes with optic fibers are used instead of LEDs. The emission spectrum of laser diode is sharper than that of LED, so that the calculation of the mean extinction coefficients over the spectrum band of the emitted light is simpler and more accurate.

In another preferred embodiment the LEDs are used with narrow-band filter to reduce the emission spectrum of the LED, so that the calculation of the mean extinction coefficients over the spectrum band of the emitted light is simpler and more accurate.

In another preferred embodiment laser diodes or LEDs for emitting light in three wavelengths are used, together with a detector, for measuring the changes in light transmission through the tissue, originating from changes in venous blood volume. Oxygen saturation in venous blood SvO₂ is then determined from the corresponding relative changes in light transmission ΔIᵥ/I_{S} for the three wavelengths, and the ratio of the relative changes in light transmission ΔIᵥ/I_{S} for the two wavelengths λ₁ and λ₂ and for the two wavelengths λ₁ and λ₃, R₁₂ and R₁₃ respectively, using Equations (13) and (15).

### BIBLIOGRAPHY

1. Mendelson Y Transcutaneous blood gas measurement. In Encyclopedia of Medical Devices and Instrumentation, J. G. Webster, Editor in Chief, John Wiley & Sons, New York, 1988 pp 448-460.
2. I Yoshiya, Y Shimady and K Tanake. Spectrophotometric monitoring of arterial oxygen saturation on the fingertip. Med Biol. Eng Comput. 18:27-32 (1980).
3. A. Babchenko, E. Davidson, Y. Ginosar, V. Kurtz, I. Feib, D. Adler and M. Nitzan, 2001. Photoplethysmographic measurement of changes in total and pulsatile tissue blood volume following sympathetic blockade. Physiol Meas. 22:389-396.
4. O. Wieben. Light absorbance in pulse oximetry In: Design of Pulse Oximeters J. G. Webster, editor. Institute of Physics Publishing, Bristol, pp 40-55, (1997)
5. P. D. Mannheimer, J. R. Casciani, M. E. Fein, S. L. Nierlich. Wavelength selection for low-saturation pulse oximetry. IEEE Trans. Biomed. Eng. 44(3): 148-158 (1997)
6. M. Nitzan, A. Babchenko, B. Khanokh, and H. Taitelbaum. The measurement of oxygen saturation in venous blood by dynamic near IR spectroscopy. J Biomed Optics. 5:155-162 (2000)
7. Schowalter J S, Calibration. In. Design of Pulse Oximeters. J. G. Webster, editor. Institute of Physics Publishing, Bristol, pp. 159-175, (1997).
8. A. Duncan , J. H. Meek, M. Clemence, C. E. Elwell. L. Tyszczuk, M. Cope and D. T. Delpy. Optical pathlength measurements on adult head, calf and forearm and the head of the newborn infant using phase resolved spectroscopy. Phys. Med Biol, 40:295-304 (1995).
9. M. Nitzan, H. de Boer, S. Turivnenko, A. Babchenko, and D. Sapoznikov 1994. Power spectrum analysis of the spontaneous fluctuations in the photoplethysmographic signal J Basic and Clinical Physiol and Pharmacol. 5:269-276.
10. M. Nitzan, S. Turivnenko, A. Milston, A. Babchenko and Y. Mahler, 1996. Low frequency variability in the blood volume and in the blood volume pulse measured by photoplethysmography. J. Biomed Optics, 1:223-229.
11. L M Seccombe, P T Kelly, C K Wong, P G Rogers, Lim S and M J Peters, 2004. Effect of simulated commercial flight on oxygenation in patients with interstitial lung disease and chronic obstructive pulmonary disease Thorax 59:966-970.

1. A method for the measurement of oxygen saturation in arterial blood, the method comprising:
(a) applying to the skin an apparatus including:
(i) light-sources for generating light of three peak wavelengths λ₁, λ₂ and λ₃; (ii) a detector arrangement; and (iii) an electronics unit, the apparatus configured to detect, for each light-source, the transmitted light through the tissue;
(b) for each light source, deriving a PPG curve;
(c) deriving from the three PPG curves a parameter related to the relative change in light transmission for each of the three wavelengths, and the ratio R₁₂ and R₁₃ of that parameter for the two wavelengths λ₁ and λ₂ and for the two wavelengths λ₁, and λ₃, respectively; and
(d) determining the oxygen saturation in arterial blood, SaO₂, from the solution of three equations including:
a. relationship between SaO₂ and R₁₂, which includes the optical path-lengths l₁ and l₂;
b. relationship between SaO₂ and R₁₃, which includes the optical path-lengths l₁ and l₃;
c. relationship between small changes in the path-length l and small changes in the wavelength λ,
where the first two equations include the values of the extinction coefficient for the three peak wavelength for oxygenated blood εₒ and for deoxygenated blood ε_{d}.

2. A method for the measurement of oxygen saturation in arterial blood, the method comprising:
(a) applying to the skin an apparatus including:
(i) light-sources for generating light of three peak wavelengths λ₁, λ₂ and λ₃; (ii) a detector arrangement; and (iii) an electronics unit, the apparatus configured to detect, for each light-source, the transmitted light through the tissue;
(b) for each light-source, determining the mean extinction coefficients over the spectrum band of the light-source emitted light for oxygenated blood εₒ and for deoxygenated blood ε_{d};
(c) for each light source, deriving a PPG curve;
(d) deriving from the three PPG curves a parameter related to the relative change in light transmission for each of the three wavelengths, and the ratio R₁₂ and R₁₃ of that parameter for the two wavelengths λ₁ and λ₂ and for the two wavelengths λ₁ and λ₃, respectively; and
(e) determining the oxygen saturation in arterial blood, SaO₂, from the solution of three equations including:
a. relationship between SaO₂ and R₁₂, which includes the optical path-lengths l₁ and l₂;
b. relationship between SaO₂ and R₁₃, which includes the optical path-lengths l₁ and l₃;
c. relationship between small changes in the path-length l and small changes in the wavelength λ,
where the first two equations include the values of said mean extinction coefficient for the three light sources for oxygenated blood εₒ and for deoxygenated blood ε_{d}.

3. A method as in claim 2, where said relationship between SaO₂ and R₁₂, which includes the optical path-lengths l₁ and l₂ is
$$Sa {O}_{2} = \frac{{ɛ}_{d 1} - {R}_{12} ( {l}_{1} / {l}_{2} ) {ɛ}_{d 2}}{{R}_{12} ( {l}_{1} / {l}_{2} ) ( {ɛ}_{02} - {ɛ}_{d 2} ) + ( {ɛ}_{d 1} - {ɛ}_{01} )}$$

4. A method as in claim 3, where said relationship between SaO₂ and R₁₃, which includes the optical path-lengths l₁ and l₃ is
$$Sa {O}_{2} = \frac{{ɛ}_{d 1} - {R}_{13} ( {l}_{1} / {l}_{3} ) {ɛ}_{d 3}}{{R}_{13} ( {l}_{1} / {l}_{3} ) ( {ɛ}_{02} - {ɛ}_{d 3} ) + ( {ɛ}_{d 1} - {ɛ}_{01} )}$$

5. A method as in claim 1, where said three peak wavelengths are in the infrared region.

6. A method as in claim 2, where said three peak wavelengths are in the infrared region.

7. A method as in claim 2, where said relationship between small changes in the path-length l and small changes in the wavelength λ is a linear relationship.

8. A method as in claim 2, where said parameter related to the relative change in light transmission is selected from the group comprising:
a. (I_{D}−I_{S})/I_{S}, where I_{D} is the maximal light transmission and I_{S} is the minimal light transmission;
b. [I(t₁)−I(t₂)]/I(t₂), where I(t₁) and I(t₂) are light transmission values at two time points, t₁ and t₂, along the rise-time of the PPG pulse,
c. ln (I_{D}/I_{S});
d. ln [I(t₁)/I(t₂)], where t₁ and t₂ are two time points along the rise-time of the PPG pulse,
e. (dI(t)/dt)/I(t) at some point along the rise-time of the PPG pulse; and
f. mean of the values of (dI(t)/dt)/I(t) at some points along the rise-time of the PPG pulse.

9. A method as in claim 1, where the light sources are selected from the group comprising: light emitting diodes, light emitting diodes with filter and laser diodes.

10. A method as in claim 2, where the light sources are selected from the group comprising: light emitting diodes, light emitting diodes with filter and laser diodes.

11. A method for the measurement of oxygen saturation in tissue venous blood, the method comprising:
(a) applying to the skin an apparatus including:
(i) light-sources for generating light of three peak wavelengths λ₁, λ₂ and λ₃; (ii) a detector arrangement; and (iii) an electronics unit, the apparatus configured to detect, for each light-source, the transmitted light through the tissue;
(b) for each light source, deriving a curve of light transmission through the tissue while venous blood volume changes;
(c) deriving from the three light transmission curves a parameter related to the relative change in light transmission for each of the three wavelengths, and the ratio R₁₂ and R₁₃ of that parameter for the two wavelengths λ₁ and λ₂ and for the two wavelengths λ₁ and λ₃, respectively, and
(d) determining the oxygen saturation in venous blood, SvO₂, from the solution of three equations including:
a. relationship between SaO₂ and R₁₂, which includes the optical path-lengths l₁ and l₂;
b. relationship between SaO₂ and R₁₃, which includes the optical path-lengths l₁ and l₃;
c. relationship between small changes in the path-length l and small changes in the wavelength λ,
where the first two equations include the values of the extinction coefficient for the three peak wavelength for oxygenated blood εₒ and for deoxygenated blood ε_{d}.

12. A method for the measurement of oxygen saturation in tissue venous blood, the method comprising:
(a) applying to the skin an apparatus including:
(i) light-sources for generating light of three peak wavelengths λ₁, λ₂ and λ₃; (ii) a detector arrangement; and (iii) an electronics unit, the apparatus configured to detect, for each light-source, the transmitted light through the tissue;
(b) for each light-source, determining the mean extinction coefficients over the spectrum band of the light-source emitted light for oxygenated blood εₒ and for deoxygenated blood ε_{d};
(c) for each light source, deriving a curve of light transmission through the tissue while venous blood volume changes;
(d) deriving from the three light transmission curves a parameter related to the relative change in light transmission for each of the three wavelengths, and the ratio R₁₂ and R₁₃ of that parameter for the two wavelengths λ₁ and λ₂ and for the two wavelengths λ₁ and λ₃, respectively; and
(e) determining the oxygen saturation in venous blood, SvO₂, from the solution of three equations including:
a. relationship between SaO₂ and R₁₂, which includes the optical path-lengths l₁ and l₂;
b. relationship between SaO₂ and R₁₃, which includes the optical path-lengths l₁ and l₃;
c. relationship between small changes in the path-length l and small changes in the wavelength λ,
where the first two equations include the values of said mean extinction coefficient for the three light sources for oxygenated blood εₒ and for deoxygenated blood ε_{d}.

13. A method as in claim 12 where said relationship between SvO₂ and R₁₂, which includes the optical path-lengths l₁ and l₂ is
$$Sv {O}_{2} = \frac{{ɛ}_{d 1} - {R}_{12} ( {l}_{1} / {l}_{2} ) {ɛ}_{d 2}}{{R}_{12} ( {l}_{1} / {l}_{2} ) ( {ɛ}_{02} - {ɛ}_{d 2} ) + ( {ɛ}_{d 1} - {ɛ}_{01} )}$$

14. A method as in claim 13, where said relationship between SvO₂ and R₁₃, which includes the optical path-lengths l₁ and l₃ is
$$Sv {O}_{2} = \frac{{ɛ}_{d 1} - {R}_{13} ( {l}_{1} / {l}_{3} ) {ɛ}_{d 3}}{{R}_{13} ( {l}_{1} / {l}_{3} ) ( {ɛ}_{02} - {ɛ}_{d 3} ) + ( {ɛ}_{d 1} - {ɛ}_{01} )}$$

15. A method as in claim 11, where said three peak wavelengths are in the infrared region.

16. A method as in claim 12, where said three peak wavelengths are in the infrared region.

17. A method as in claim 12, where said relationship between small changes in the path-length l and small changes in the wavelength λ is linear relationship

18. A method as in claim 12, where said parameter related to the relative change in light transmission is selected from the group comprising:
a. (I_{D}−I_{S})/I_{S}, where I_{D} is the maximal light transmission and I_{S} is the minimal light transmission;
b. [I(t₁)−I(t₂)]/I(t₂), where I(t₁) and I(t₂) are light transmission values at two time points, t₁ and t₂, along the rise-time of the PPC pulse;
c. ln (I_{D}/I_{S});
d. ln [I(t₁)/l(t₂)], where t₁ and t₂ are two time points along the rise-time of the PPG pulse;
e. (dI(t)/dt)/I(t) at some point along the rise-time of the PPG pulse; and f mean of the values of (dI(t)/dt)/I(t) at some points along the rise-time of the PPG pulse.

19. A method as in claim 11, where the light sources are selected from the group comprising: light emitting diodes, light emitting diodes with filter and laser diodes.

20. A method as in claim 12, where the light sources are selected from the group comprising: light emitting diodes, light emitting diodes with filter and laser diodes.

21. A method as in claim 12, where said change in venous blood volume is achieved by closing the veins conveying blood from said tissue, by use of a pressure cuff.

22. A method as in claim 12, where said change in venous blood volume is spontaneous.

23. A method as in claim 22, where said spontaneous change in venous blood volume is at the respiratory rate.

24. A method as in claim 22, where said spontaneous change in venous blood volume is at a frequency of about 0.1 Hz

25. A method as in claim 22, where said spontaneous change in venous blood volume is at a frequency of 0.01-0.04 Hz.
